(19) United States
(12) Patent Application Publication
(10) Pub. No.: US 2025/0231189 A1
(43) Pub. Date: Jul. 17, 2025

(54) MEASURING FREQUENCY OF PATHOGEN-SPECIFIC T CELLS IN PERIPHERAL BLOOD AS ESTABLISHED BY TCR-INDUCED CA(2+) SIGNALING

(71) Applicant: Thomas Jefferson University, Philadelphia, PA (US)

(72) Inventors: Yuri Sykulev, Newtown Square, PA (US); Nadezhda Anikeyeva, Bryn Mawr, PA (US)

(21) Appl. No.: 19/058,712

(22) Filed: Feb. 20, 2025

Related U.S. Application Data
(63) Continuation of application No. 16/612,675, filed on Nov. 11, 2019.
(60) Provisional application No. 62/505,667, filed on May 12, 2017.

(51) Int. Cl. G01N 33/58; G01N 33/50; G01N 33/84

(52) U.S. Cl. CPC G01N 33/582; G01N 33/505; G01N 33/84; G01N 2800/52

(57) ABSTRACT

A method for measuring kinetics of Ca²⁺ flux in differentially responding T cells that form monolayer on the glass surface in response to antigenic peptides or live target cells comprising: immobilizing T cells labeled with Ca²⁺ sensitive fluorophore on the glass bottom of a well, covered with capturing antibody or a capturing protein that bind to non-stimulatory T-cell surface receptor; adding to the well a single or multiple peptide epitopes that binds to the cell surface MHC molecules to be presented for recognition by cognate T cells; the stimulatory signal could also be delivered by live target cells that display peptide epitope(s); wherein the recognition of stimulatory of pMHC by the peptide specific T cells leads to increase of intracellular Ca²⁺ level and fluorescence intensity in the responding T cells, which is then identified after the subtracting fluorescence intensity for every T cell before and after the addition of the peptide antigens; scoring each responding T cell into a category according to three categories including: a rapid and sustained T-cell response, an oscillatory response, or a delayed and oscillatory response; and measuring changes in number of an individual T cells with increased intracellular fluorescence as function of time provides the kinetic curve of the TCR-mediated Ca²⁺ signaling.

### PRIORITY CLAIM

This application claims the benefit of U.S. Provisional Application Ser. No. 62/505,667 filed May 12, 2017, the disclosure content of which is hereby incorporated by reference in its entirety.

### FIELD OF INVENTION

The present application is generally related to methods for evaluating the frequency of various subsets of pathogen-specific and tumor-specific T cells that are characterized by a pattern of Ca²⁺ signaling mediated by productive engagement of antigen-specific receptor, reflecting the effectiveness of immune responses.

### BACKGROUND OF THE INVENTION

Currently, determining the frequency of T cells with specificity of interest is performed by staining of the T cells with the pMHC/tetramers or intracellular staining for cytokine or ELISpot assays. While these processes have some ability to determine specificity of T-cells, each has significant limitations. Staining with the tetramers does not allow determining functional capacity of virus-specific T cells. ELISpot assay and intracellular cytokine staining provide information about functional activity of T cells, but does not detect antigen-specific cells that are unable to produce the indicator cytokine. In addition, ELISpot assay takes at least 24 hrs. to complete, a time during which T cells are exposed to stimulation resulting in activation of initially antigen-inexperienced T cells contributing to potentially pseudo positive data.

Several United States patents or publications, or other literature have proposed approaches to characterize frequency of T-cells with the specificity of interest. However, these approaches are generally lacking in one or more features and do not evaluate the frequency of pathogen-specific T cells with different efficiencies.

US Pub. No. 2015/0030533—entitled “Compositions and methods for the detection diagnosis and therapy of hematological malignancies” proposes methods for eliciting immune and T cell responses to specific malignancy-related antigenic polypeptides. The '533 Publication, however, does not address quantifying the frequency of T cells through measurement of intracellular Ca²⁺ in individual T Cells as in a new method defined herein.

US Pub. No 2007/0059845—entitled “Reagents for the detection of protein phosphorylation in T-cell receptor signaling pathways” is related to phosphorylation sites downstream of the T-cell receptor that provides for selective detection and quantification of phosphorylated proteins. However, there is no mention of detection strategies incorporating measurements of Ca²⁺ in individual T cells as in the new methods described herein.

Altman, Moss, Goulder, et. al.: “Phenotypic Analysis of Antigen-Specific T Lymphocytes”; Science 274 (5284): 94-96; addresses a tetramer assay to detect and quantify T-Cells that are specific for a given antigen within a blood sample. However, the Altman publication does not detect via Ca²⁺ in individual T cells as in the methods disclosed herein.

Indeed, US Pub. Nos '533 and '845, appear to generally describe the field regarding detection of T-cells and related signaling pathways. However, neither the '533 nor the '845 Publication provided any disclosure of measurement of calcium ions as a mechanism for identifying specific or individual T cells and thus fail to provide mechanisms for detection and quantification of virus specific T-cells.

The Altman publication is related to Tetramer technology, which is described in this disclosure. However, the Tetramer technology does not measure Ca²⁺ signaling, and instead measures TCR specificity. Accordingly, this assay utilizes a completely different detection strategy.

Our recent patent application no. PCT/US16/39313; entitled “MEASURING FREQUENCY OF PATHOGEN-SPECIFIC T CELLS IN PERIPHERAL BLOOD” proposes a method of detection of pathogen and cancer-specific in a polyclonal population of peripheral blood T cells based on measuring frequency of responding T cells and the kinetics of Ca2+ signaling that provide an information regarding the efficiency of T cell response. However, different T cells in polyclonal T population display various pattern of Ca²⁺ mobilization indicative of different potencies of T cells. Averaging of these parameters are not always useful, especially at the conditions of highly variable T cell response.

Therefore, there is a need for new strategies for analyzing and quantifying various pathogen and cancer-specific T cells subsets with different pattern Ca2+ responses. Through this new methodology, we can better characterize and compare the efficiency of polyclonal T cell populations with the same specificities allowing to reveal a much better correlation with clinical outcomes of pathogen infections and cancer.

### SUMMARY OF THE INVENTION

In accordance with these and other objects, a first embodiment of an invention disclosed herein is directed to a method of identification of pathogen-specific T cells with the same specificity but having distinct patterns of TCR-mediated Ca²⁺ signaling as well as the frequency of these T cells.

The embodiments described herein allow for identification of various patterns of Ca²⁺ flux in responding to antigenic peptides T cells that form monolayer on the glass surface. Freshly isolated T cells labeled with Ca²⁺ sensitive fluorophore are immobilized on the glass bottom of a well-covered with non-stimulatory antibody specific for a cell surface receptor. Peptides of interest that are added to the T cell monolayer bound to the MHC molecules presented for recognition by cognate T cells. The recognition of stimulatory of pMHC by T cells specific for the MHC-bound peptide leads to increase of Ca²⁺ and fluorescence intensity in the responding T cells, which could then be identified after the subtracting fluorescence intensity for every T cell before and after the addition of the peptide antigens.

In a further embodiment, a method comprises coating a glass bottom plates with Poly-L-Lysine and, after washing free of unbound reagents, the plates were covered with antibody specific for receptor on the surface T cells that do not interfere with T cell responses. We utilize cloned T cell or freshly purified T cells from donor's PBMC labeled with Ca²⁺ sensitive fluorophore. The capturing of the T cells by the immobilized antibody was facilitated by brief centrifugation at 200 g and unbound cells were removed by gentle washing. We analyze the quality of the T-cell monolayer, which formed on the glass surface, and measured background intracellular fluorescence for every cell by means of wide field fluorescent microscopy. We then add to the wells an antigenic peptide or a set of peptides of interest to be tested and measure fluorescence intensity for every cell in the same fields as before and after at various time points with interval of 20-30 seconds. Stimulating cells with ionomycin and non-stimulatory peptides serve as positive and negative controls, respectively. Changes of the fluorescent intensity in individual cells before and after peptide injection are recorded as a function of time using MetaMorph software. The fractions of responding cells having different pattern of changes in fluorescent intensity with time have been determined. This leads to establishing a pattern of pathogen-specific response providing information regarding frequencies of the T cells with various patterns of Ca fluxes that are linked to different efficiencies of T-cell responses. We have identified three groups of responding CD8+ T cells: (i) T cells that rapidly develop sustained Ca²⁺ signaling typical for T-cell responses to a strong agonist peptide, (ii) T cells developing delayed Ca²⁺ signaling that is characteristic for T-cell responses to a weak agonist peptide, and (iii) T cells that revealed rapid oscillating Ca²⁺ signaling indicative of suboptimal T cell responses. T cells showing sporadic and transient Ca²⁺ spikes were considered unresponsive. See FIG. 1.

These data allow us to determine fractions of pathogen-specific T cells with distinct abilities to respond to a pathogen in donor's PBMC.

The invention of a novel approach allows detecting, quantification of the frequency of pathogen-specific T cells of various potencies to multiple antigenic peptide epitopes. The Assay measures time-dependent changes in intracellular Ca²⁺ signaling in individual T cells. T cells are labeled with Ca²⁺ sensitive fluorophore and are placed on the glass bottom of a well-covered with antibodies against non-stimulatory T cells' surface receptors. A peptide antigen is injected into the well and the peptide binds to MHC molecules on the T-cell surface. Changes in the intracellular concentration of Ca²⁺ in responding T cells leads to changes in intracellular fluorescence over time that is detected by fluorescent microscope.

This approach suitable for analyzing frequency of T cells of various quality recognizing either pathogen-specific or tumor-associated antigens derived from peripheral blood or extracted from tumor (tumor infiltrating lymphocytes or TIL) using either pathogen-or tumor-associated peptide epitopes or antigen-presenting cells sensitized with these epitopes or live tumor cells. In addition, to measuring frequency and quality of various subsets of CD8 T cells with desired specificity, the frequency and quality of various CD4 T cell subsets recognizing peptide-MHC-II ligands can be also measured.

A preferred embodiment is directed towards an assay for detecting and quantification of the frequency of T cells to multiple antigenic peptide epitopes wherein the Assay measures intracellular Ca²⁺ signaling in individual T cells; the T cells are labeled with Ca²⁺ sensitive fluorophore and are placed on the glass bottom of a well-covered with antibodies against non-stimulatory T cells' surface receptors; A peptide antigen is injected into the well and the peptide binds to MHC molecules on the T-cell surface; an Increase in the intracellular concentration of Ca²⁺ in responding T cells leads to rise in cell fluorescence that is detected by fluorescent microscope; wherein the responding T cells are differentiated into at least three categories; and the response is calculated for each of the three categories of cells.

In a preferred embodiment, wherein the at least three categories including a rapid and sustained T-cell response, an oscillatory response, or a delayed and oscillatory response. In a preferred embodiment, a fourth category which is a non-responsive T cell.

A preferred embodiment is directed towards a method for detection of the frequency of different responding T cells, each responding to multiple antigenic peptide epitopes comprising: coating glass surface with an agent capable to bind either an antibody or other capturing proteins; covering the surface with an antibody or capturing proteins that binds to a receptor on T-cell surface without interfering with Ca²⁺ flux; adding cloned or polyclonal T cells or alfa/beta T cells labeled with Ca²⁺ sensitive fluorophore to the surface to generate monolayer of the T cells; taking first image of the T-cell monolayer to determine a level of background fluorescence in every individual cell; determining a classification for each responding T cell according to one of four response curves; adding a single or multiple peptide epitopes or live target cells presenting potential peptide epitopes to the T-cell monolayer; measuring the level of fluorescence in every individual T cells on the monolayer by taking second image of the T-cell monolayer followed by peptide(s) or live target cells addition bearing peptide epitope; quantifying responses of individual T cells in each of the four classes, by subtracting intracellular fluorescence measured after taking the first image from that acquired after the second image; and averaging the response in each of the four classes to generate a response rate for each of the four classes.

A preferred embodiment is directed towards a method for measuring kinetics of Ca²⁺ flux in differentially responding T cells that form monolayer on the glass surface in response to antigenic peptides or live target cells comprising: immobilizing T cells labeled with Ca²⁺ sensitive fluorophore on the glass bottom of a well, covered with capturing antibody or a capturing protein that bind to non-stimulatory T-cell surface receptor; adding to the well a single or multiple peptide epitopes that binds to the cell surface MHC molecules to be presented for recognition by cognate T cells; the stimulatory signal could also be delivered by live target cells that display peptide epitope(s); wherein the recognition of stimulatory of pMHC by the peptide specific T cells leads to increase of intracellular Ca²⁺ level and fluorescence intensity in the responding T cells, which is then identified after the subtracting fluorescence intensity for every T cell before and after the addition of the peptide antigens; scoring each responding T cell into a category according to FIG. 1; and measuring changes in number of an individual T cells with increased intracellular fluorescence as function of time provides the kinetic curve of the TCR-mediated Ca²⁺ signaling.

A preferred embodiment is directed towards a method for calculating the number of responding T cells, having a particular respond pattern, in a sample comprising: coating glass bottom surface of 96-well plates with an agent capable to bind either an antibody or other capturing proteins; washing said plates free of unbound reagents, wherein the plates were covered with an antibody or other capturing proteins specific for non-stimulatory receptor on the T-cell surface that do not interfere with the induction of T-cell response; blocking the plates with BSA solution; capturing cloned T cell or freshly purified T cells from donor's PBMC labeled with Ca²⁺ sensitive fluorophore; measuring background of intracellular fluorescence for every cell by means of wide field fluorescent microscopy; adding to the wells an antigenic peptide of interest or live target cell presenting potential peptide epitope; measuring fluorescence intensity for every cells in the same fields before and after addition of the stimuli at several time points; stimulating cells with ionomycin and non-stimulatory or “self” peptides serve as positive and negative controls, respectively; calculating the number of cells responding to one of four categories: a rapid and sustained T-cell response, an oscillatory response, a delayed and oscillatory response, or a non-responsive cell; and comparing intracellular fluorescence in individual cells before and after peptide or live target cells injection using MetaMorph software wherein the number of cells that remain fluorescent in each analyzed field are quantified to calculate the total number of the responding cells per 10⁶ cloned T cells or donor's PBMC.

A preferred embodiment is directed towards a method to characterize cell surface markers on T cells with the specificity of interest in order to determine a stage of T-cell differentiation comprising: Immobilizing freshly isolated CD8 T cells labeled with Ca²⁺ sensitive fluorophore and antibodies labeled with non-overlapping fluorophore against cell surface markers of interest on the glass bottom of a well, covered with capturing antibody or a capturing protein that bind to non-stimulatory T-cell surface receptor; measuring background intracellular fluorescence for every cell of the T-cell monolayer and detecting individual T cells that express cell surface markers of interest by means of wide field fluorescent microscopy; calculating total number of cells having an expression pattern according to one of three categories including rapid and sustained T-cell response, an oscillatory response, or a delayed and oscillatory response, that express cell surface markers of interest per 10⁶ CD8 T cells; adding to the wells an antigenic peptide(s) to be tested; comparing intracellular fluorescence in individual cells before and after peptide injection using MetaMorph software wherein the number of cells that remain fluorescent in each analyzed field are quantified to calculate the total number of the responding CD8 T cells per 10⁶ cells; and calculating the fractions of responding cells in each of the three categories that do or do not express surface markers of interest.

A preferred embodiment is directed towards a method for measuring the frequency of responding T cells with the specificity of interest using live target cells presenting peptide(s) of interest or nanoparticles carrying soluble peptide-MHC ligands or any other peptide-MHC oligomers to stimulate T cells recognizing these ligands comprising: Immobilizing T cells labeled with Ca²⁺ sensitive fluorophore on the glass bottom of a well, covered with capturing antibody or a capturing protein that bind to non-stimulatory T-cell surface receptor; measuring background of intracellular fluorescence for every cell of the T-cell monolayer by means of wide field fluorescent microscopy; adding to the wells live target cell presenting peptide(s) of interest or nanoparticles bearing various peptide-MHC or any other peptide-MHC oligomers that ought to be tested; measuring fluorescence intensity for every cells in the same fields after the exposure of T cells in the T-cell monolayer to the above stimuli; determining a category for response for each of the responding cells, according to one of three categories, including rapid and sustained T-cell response, an oscillatory response, or a delayed and oscillatory response; comparing intracellular fluorescence in individual cells before and after the stimulation using MetaMorph software wherein the number of cells that remain fluorescent in each analyzed field are determined as responding cells; and calculating the total number of each type of the responding cells per 10⁶ cloned T cells or donor's PBMC.

A preferred embodiment is directed towards a method to determine the frequency and functional activity of each of four types of antigen-specific CD8 T cells from human PBMC through an assay based on measurement of T-cell intracellular Ca²⁺ signaling induced in response to antigen recognition by T-cell receptor comprising: immobilizing freshly isolated CD8 T cells from human PBMC either intact or labeled with Ca²⁺ sensitive fluorophore on the glass bottom of a well, covered with capturing antibody or a capturing protein that bind to non-stimulatory T-cell surface receptor; adding to the wells unlabeled or fluorescently labeled peptide-MHC proteins assembled on nanoparticles or any other peptide-MHC oligomers to detect antigen-specific T cells and/or to induce Ca²⁺ signaling in the responding T cells; wherein the recognition of unlabeled stimulatory pMHC by the specific T cells labeled with Calcium fluorophore leads to increase of intracellular Ca²⁺ level and fluorescence intensity in the responding T cells, determine which of four classes the responding cells belong to: including a rapid and sustained T-cell response, an oscillatory response, a delayed and oscillatory response, or a non-responding cell, and subtracting fluorescence intensity for every T cell measured before the addition of the stimulatory peptide-MHC oligomers. The binding of fluorescently labeled cognate pMHC to unlabeled T cells will identify both responding and non-responding T cells specific for the same peptide-MHC ligands; and calculating a fraction of responding T cells with the specificity of interest.

A preferred embodiment is directed towards a method allowing measurement of kinetics of Ca²⁺ flux in responding to antigenic peptides on T cells that form monolayer on the glass surface of a variety of differentially responding T cells; comprising: fixing freshly isolated T cells labeled with Ca²⁺ sensitive fluorophore are immobilized on the glass bottom of a well-covered with non-stimulatory antibody specific for a cell surface receptor.; adding Peptides of interest that are added to the T cell monolayer bound to the MHC molecules presented for recognition by cognate T cells; increasing the recognition of stimulatory of pMHC by the peptide specific T cells leads increase of Ca²⁺ and fluorescence intensity in the responding T cells, which could then be identified after the subtracting fluorescence intensity for every T cell before and after the addition of the peptide antigens; measuring the number of responding cells as a function of time characterize the kinetics of the Ca²⁺ flux in responding T cells; characterizing the responding T-cells according to one of four classes of response, so as to determine a class of each cells; plotting a time plot to determine the kinetics of the Ca²⁺ response for a particular antigen and to determine certain responding cells having characteristics that are suitable for use as therapeutic stem cells for a patient.

A preferred embodiment is directed towards a method of calculating the response rate of a T cells comprising: coating a glass bottom plates with Poly-L-Lysine and, after washing free of unbound reagents, the plates were covered with antibody specific for non-stimulatory receptor on the surface T cells that do not interfere with T cell responses; blocking the plates with BSA solution prior to addition of T cells. We utilize cloned T cell or freshly purified T cells from donor's PBMC labeled with Ca²⁺ sensitive fluorophore; capturing of the T cells by the immobilized antibody was facilitated by brief centrifugation at 200 g and unbound cells were removed by gentle washing; analyzing the quality of the T-cell monolayer, which formed on the glass surface, and measured background intracellular fluorescence for every cell by means of wide field fluorescent microscopy; adding to the wells an antigenic peptide of interest to be tested and measure fluorescence intensity for every cells in the same fields as before at several time points; stimulating cells with ionomycin and non-stimulatory or “self” peptides serve as positive and negative controls, respectively; determining the class of cell response according to FIG. 1 (a rapid and sustained T-cell response, an oscillatory response, a delayed and oscillatory response, a non-responding cell); comparing cellular fluorescence in individual cells before and after peptide injection using MetaMorph software; quantifying the number of cells that remain fluorescent in each analyzed field and to calculate the total number of the responding cells per 10⁶ cloned T cells or donor's PBMC.

A preferred embodiment is directed towards a method for detection of the frequency of T cells to multiple antigenic peptide epitopes comprising: Coating a well with poly-L-Lysine or, in further embodiments, optically clear plastic surface can be used that is modified with other chemical agents capable to bind antibodies or other capturing proteins; capturing TS2/4 antibody with said poly-L-Lysine, or in further embodiments, streptavidin can be utilized to capture biotinylated antibody. Furthermore, any other capturing molecules specific to T cell's surface that do not interfere with Ca2+ flux can be utilized; thereafter, adding cloned CD8 T cells with known specificity (OR polyclonal CD8 T cells) and labeling each with Ca²⁺ fluorophore Fluo-4 and adding the T cells to the wells, or, in further embodiments, other cells, including CD4 T cells or gamma/delta T cells can be added to the wells, or, in further embodiments, the cells can be labeled with any Ca2+ sensitive fluorophore and wherein changes in bioelectric properties of T cells can be measured. Finally, you measure the fluorescence and determine classes of response based on the rate and pattern of response of the responding cells. This information can then be utilized to determine whether a particular cell line has the predetermined proper response rate for a particular treatment.

A preferred embodiment is directed towards a method for determining the efficiency of pathogen-specific T cells comprising: preparing a continuous monolayers of freshly isolated T cells labeled with Ca2+ sensitive fluorophore; adding a suspension of tumor cells could be used to detect tumor specific T cells within the monolayer; measuring Ca2+ responding T cells in the monolayer and to measure the kinetics of Ca2+ flux; and determining the frequency and efficiency of pathogen-specific or tumor-specific T cells within the monolayers.

A preferred embodiment is directed towards a method for predicting efficacy of a treatment and a clinical outcome comprising: analyzing the frequency and the efficiency of the responding T cells; wherein said frequency and efficiency will provide an essential information regarding status of the immune response against pathogens or cancer in order to predict the outcome of the infection or cancer spread as well as to choose appropriate treatment for tested individuals; wherein the latter will have significant impact on the cost of treatment and will increase survival rate of the patients; charactering the response rate of each responding cell into one of at least four groups; averaging the response of the cells in each group as compared to the control; plotting the response of each group over time; wherein an efficiency within one standard deviation of the control indicates a functioning immune system; and wherein an efficiency is reduced by more than one standard deviation of the control indicates a compromised immune system.

Additional features and embodiments will be apparent to one of ordinary skill in the art upon consideration of the following detailed description of preferred embodiments and descriptions of the best mode of carrying out the invention.

### BRIEF DESCRIPTION OF THE DRAWINGS

FIG. 1. Depicts various pattern of TCR-mediated Ca2+ signaling in T cells: (i) rapidly developed sustained Ca²⁺ signaling (top left); (ii) delayed but sustained Ca²⁺ signaling that is characteristic for T-cell responses to a weak agonist peptide (top right), (iii) oscillating Ca²⁺ signaling indicative of suboptimal T cell responses (bottom left); (iv) sporadic and transient Ca²⁺ spikes that are characteristic of unresponsive T cells (bottom right).

FIG. 2. Depicts various distribution of various fractions of peripheral human CD8 T cells from normal individual that are specific for human cytomegalovirus (HCMV) and are characterized by distinct patterns of TCR-mediated Ca2+ signaling.

FIG. 3. Demonstrate changes in the distribution of various fractions of peripheral human CD8 T cell specific for human cytomegalovirus (HCMV) that were derived from peripheral blood of a patient who underwent bone marrow transplantation. The changes in the fractions of efficient and less efficient HCMV-specific CD8 T cells taken at distinct time-points are consistent with the HCMV reactivation and the ability of the patient to control HCMV infection.

FIG. 4. Rapid Ca2+ mobilization in cloned T cells (68A62) in response to a strong agonist peptide. Majority of the cells (about 90%) revealed strong and sustained pattern of Ca2+ flux.

FIG. 5. Slow Ca2+ mobilization in cloned T cells (68A62) in response to a weak agonist peptide. Majority of the cells show delayed and oscillating Ca2+ response.

FIG. 6. Ca2+ mobilization in cloned T cells (68A62) in response to suboptimal concentration of a strong agonist peptide. Majority of the cells demonstrated rapid and oscillating pattern of Ca2+ mobilization.

### DETAILED DESCRIPTION OF THE PREFERRED EMBODIMENTS

Accordingly, the methods as described in the embodiments herein are directed towards methods of measuring the response of intracellular calcium in T cells, where we differentiate between the responses of individual cells.

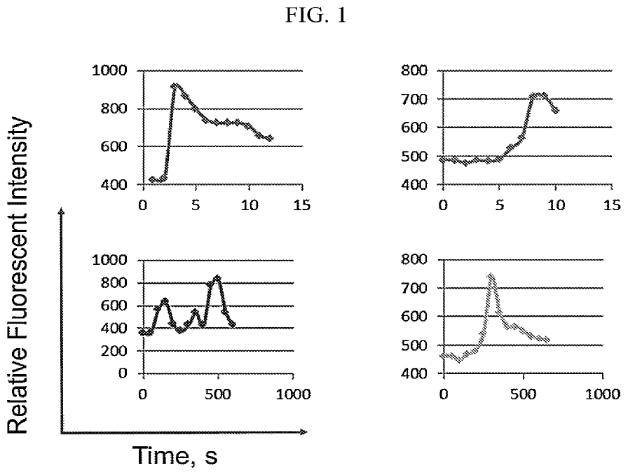

FIG. 1 details four patterns of TCR-mediated Ca2+ signaling in T cells: (i) rapidly developed sustained Ca²⁺ signaling (top left); (ii) delayed Ca²⁺ signaling that is characteristic for T-cell responses to a weak agonist peptide (top right), (iii) oscillating Ca²⁺ signaling indicative of suboptimal T cell responses (bottom left); and (iv) sporadic and transient Ca²⁺ spikes that are characteristic of unresponsive T cells (bottom right).

These various categories of T cells are characterized by pattern of Ca2+ mobilization. Because Ca2+ mobilization, i.c., increase the level of intracellular Ca2+ in responding T cells, occurs in time, there are various dependencies of the Ca2+ concentration versus time. Various patterns of these changes are shown in FIG. 1.

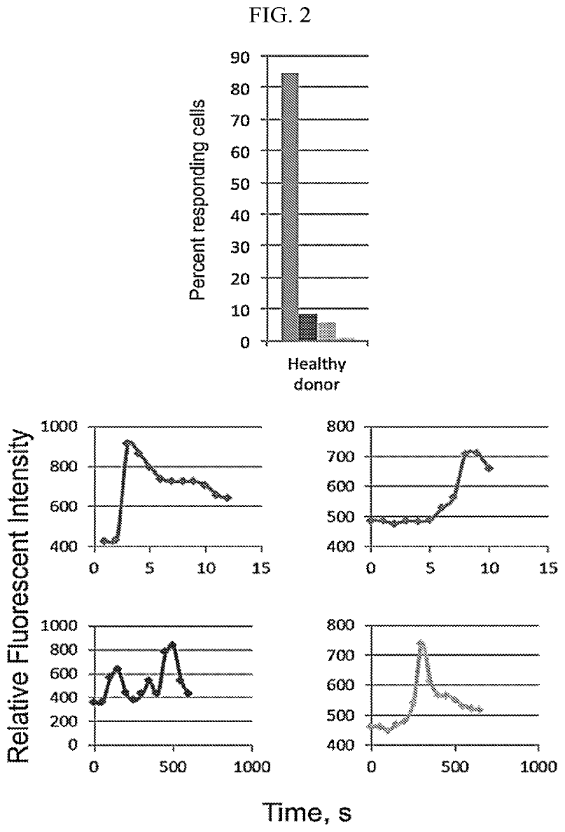

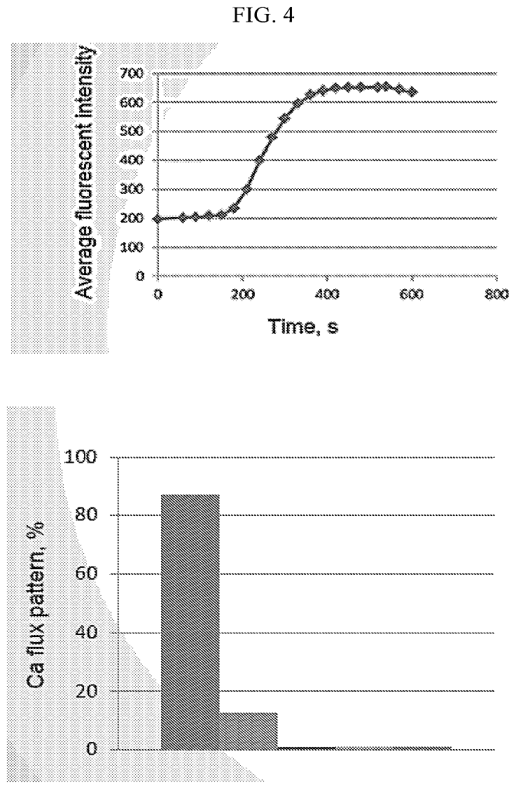

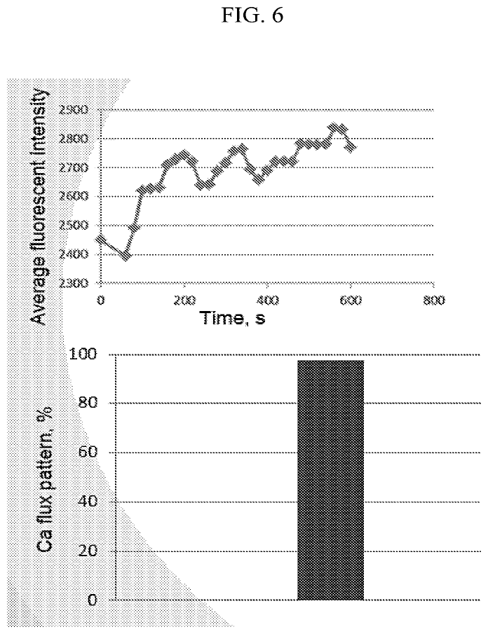

With this in mind, we have identified several patterns of Ca2+ mobilization in responding T cells. (see FIG. 2). First, very rapid and sustain mobilization of intracellular Ca2+ (top left). Second, oscillation of intracellular Ca2+ (bottom left). Third, delayed Ca2+ mobilization (top right). Forth, spontaneous and transient Ca2+ mobilization (bottom right). Thus, for each sample, we could determine fraction of T cells that are characterized by a particular pattern of Ca2+ mobilization. To provide experimental evidence that link distinct pattern of Ca2+ mobilization with a particular quality of T cell response, we have applied this approach to analysis fractions of T cells within CTL clones responding to pMHC ligands with different potency and found that even within T-cell clone individual T cells may respond to the same ligand with different potency. More than 80% of HIV-specific T cells showed very rapid and sustained response to a strong agonist ligand, while less than 20% revealed slower Ca2+ mobilization (FIG. 4). Very small fraction of T cells exposed oscillating or spontaneous responses. Responses of the same T cells to a weak agonist pMHC ligand exhibit very slow kinetics of Ca2+ mobilization and very different pattern of Ca2+ responses where majority of the T cells have oscillating pattern Ca2+ response (FIG. 6). T cell response to a strong agonist pMHC ligand at much lower concentration revealed slow oscillating kinetics of Ca2+ mobilization and oscillating pattern of Ca2+ response by the majority of responding T cells (FIG. 6). These data demonstrate that analysis of the distribution of various fraction of responding T cells could provide an important information regarding the quality of T cells response against virus infected or cancer cells.

Various patterns of Ca2+ responses may be also utilized to characterized responses of T cells at various stages of differentiation. For instance, highly effective T cells (those are capable to exercise very strong responses, but are at the final stage of differentiation and do not proliferate well) are characterized by very rapid increase of intracellular Ca2+ which is sustained for a relatively long time (Top Left FIG. 1). T cells that can proliferate well but are not very good effectors are characterized by oscillating pattern (bottom left—FIG. 1). Although these T cells are less effective they could further differentiate into highly effective T cells (top left FIG. 1) and could expend very well. Our data show that donors of bone marrow who have more cells with bottom left (oscillating pattern) would better perform in a recipient. Such T cells have a higher potency to expend in recipient body and could develop into highly efficient cytolytic effectors. Accordingly, the methods herein could be used to evaluate a donor for bone marrow transplant. Identification of a donor having both the proper compatibility and also a high number of T cells having a higher potency to expand in the recipient body would increase the likely hood of success of the transplant. Thus, by performing this analysis on one or more donors, one can evaluate and provide information that can contribute to the selection of an optimal donor for bone marrow transplantation.

In addition, we chose to analyze the kinetics of Ca2+ mobilization in responding T cells for two principal reasons: (i) changes in intracellular Ca2+ regulates about 80% genes in T cells and (ii) we have previously shown that kinetics of Ca2+ mobilization in T cells regulates delivery of cytolytic granules to target cells and accounts for the kinetics of target cell destruction by cytotoxic T lymphocytes.

In our prior publications, we first proposed to analyze kinetics of Ca2+ mobilization in population of polyclonal T cells averaging time-dependent increased in the intracellular Ca2+ for different T cells in the polyclonal population. While our analysis has proved to be useful, we have realized that averaging Ca2+ responses could be misleading. This became clear from the analysis of Ca2+ mobilization in CTL clone 68A62 stimulated with pMHC ligands of different potency. This mimics T-cell responses of different potencies within a polyclonal population of T cells. Thus, average time dependent Ca2+ mobilization in a T cell population containing relatively small fraction of highly efficient T cells and a larger fraction of much less efficient T cells could be indistinguishable from a population of T cells with just average potency. Meanwhile, changes of the ratio of highly efficient and less efficient T cells could provide very important information on the dynamic of the immune response against virus or cancer.

Thus, the various ligands provided to the cells show that different populations of cells have different response to strong or weak agonist peptides. Thus, where there is a high population of strongly responding cells, lower potency therapeutics (agonists) might be suitable as compared to a stronger agonist needed to control HCMV or other virus within the patient.

The characteristics of the responding cells is highly variable. After immobilizing (attaching) cells to a plate and taking initial images to determine background, T cells can be stimulated to respond through the Ca2+ flux methods described herein. However, the characteristic and variety of the response is highly variable.

Our data show that certain responses are indicative of more successful transplant candidates, as one example, or cells that will have a stronger response in a long term, as necessary. Accordingly, through the methods described herein, we cannot only see the entirety of the response, through averaging of the response, but we can create a better and more precise response and fit to our particular needs by placing the responding cells into a best fit of four categories.

When selecting a donor, or identifying response, we recognize that a health donor, or where there is an absence of CMV, or control of CMV will have a response rate that is similar to that as depicted in FIG. 2. Indeed, FIG. 2 specifically indicates a healthy donor, wherein more than 80% of the T cells are responding with the top left graph. Majority of healthy individuals are infected by HCMV, but successfully control the virus. Thus, the pattern of Ca2+ mobilization presented in FIG. 2 clearly indicates that T cells showing rapid and sustain Ca2+ mobilization are efficient virus-specific T cells capable of controlling the virus. Yet, despite this response, we recognize that there are variations in response, both over time and also to concentrations of an antigen.

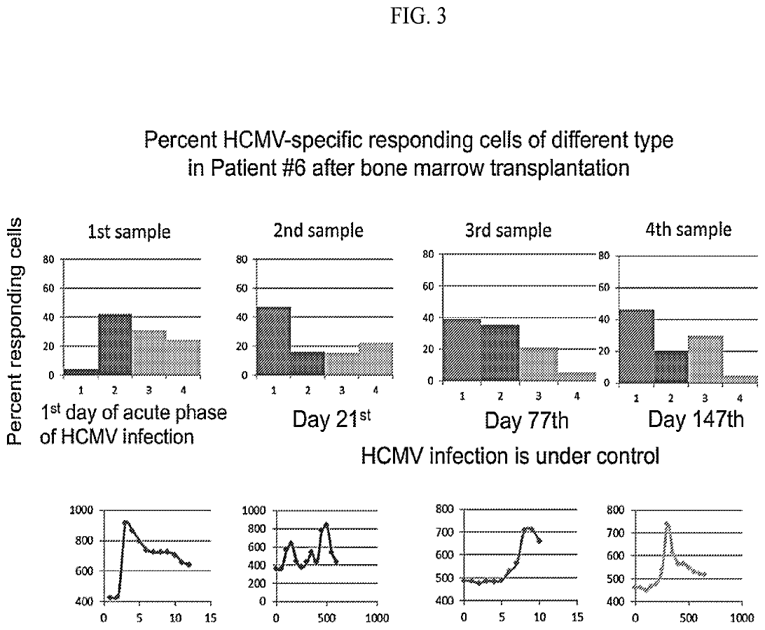

For example, in FIG. 3, we show that a response to CMV changes in the patient over time, as the patient becomes under control of the CMV infection, post-transplant. Therefore, if we simply take an average of all responding cells, we may not find a significant difference in the total response of the T cells, however, review of the changes over time, show significant differences in the actual response, the types of cells responding, and their Ca 2+ response patterns.

FIG. 3 further develops this concept. A patient is immuno compromised and has a CMV infection that is not controlled. The patient is subjected to a bone marrow transplant from a health donor who was HCMV negative (or under control). Four graphs are identified with the percentage of responding cells, as corresponding to each of the four classes of response we have identified. The response curves are provided at the bottom with the first being a good response, second oscillation, third a slow or poor response, and 4^{th} a transient or unresponsive T-cell. Then the samples are provided at Day 35 (1^{st} day of acute phase of hCMV infection), Day 56 (day 21 of infection), Day 112 (day 77 of infection), and Day 182 (day 147 of infection) after bone marrow transplantation. The response level of the T-cells is indicative of the quality of management of the HCMV infection, namely, based on the quality of response, we can expect to have more or less control of the HCMV infection.

The graphs are clear in the change over time in response to the CMV infection. In the first sample, few cells are responding well, and most cells are poorly responding, either oscillating, or a delayed response, or an acute spike (non-responsive). The patient is not yet identifying the response in T-cells to someone that is controlling the infection and thus the patient remains in a state where the CMV is not in control.

The second sample (titled Day 21) shows a very different cell response with a large percentage of the cells quickly and sustainably responding, and the CMV is under control. This pattern of control continues through the rest of the tests at day 77 and day 147. The transplant, with regard to CMV, is successful because at day 147 the CMV is under control.

FIG. 4 details a response of CD8 CTL to a strong agonist peptide 10-4 M IV9 peptide. The response of majority of T cells is rapid and sustaine; despite 20% of T cells showing pattern of Ca2+ mobilization of inefficient T cells, average dynamics of Ca2+ mobilization revealed rapid and sustained response as well.

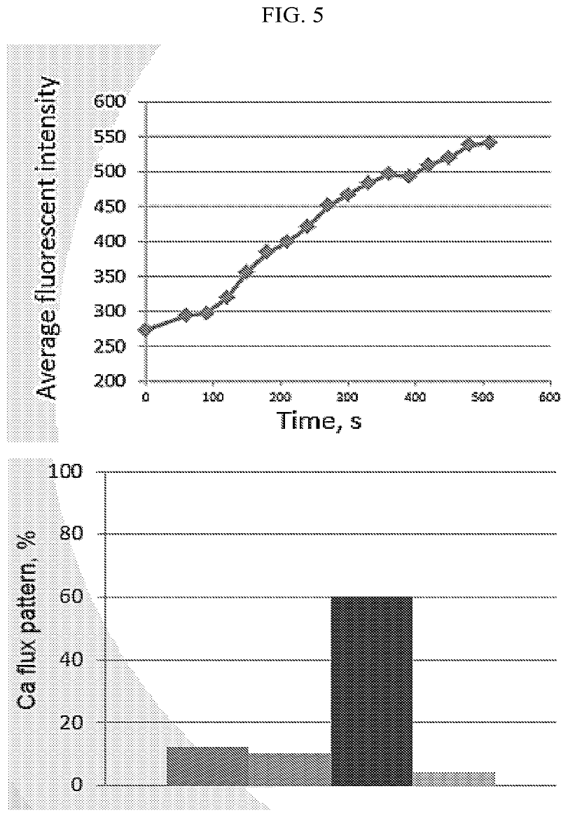

FIG. 5 depicts a different peptide than was used in FIG. 4. The average kinetics of Ca2+ mobilization is slow and majority of T cells show oscillating pattern of Ca2+ mobilization indicative of inefficient response. Such cells would not be a good target for a donor, or would identify that the cells are not responding well to the agonist provided.

FIG. 6 depicts the same peptide from FIG. 4, but in a reduced concentration. This is a response T cells clone to a viral peptide that is a strong agonist peptide; but only a small number of peptide was added to induce T cell response accounting for a lower strength of T cells stimulation that is materialized in a rapid kinetics of Ca2+ mobilization and oscillating pattern of Ca2+ response indicative of inefficient T cell response. We see that the response of CA flux is quick, but oscillating. Thus, even though this is a strong peptide, the amount is not sufficient to induce a strong response. Thus, to get a better response, we would need to increase the concentration of the peptide provided to the cells.

Accordingly, one embodiment is directed to an assay for detecting and quantification of the frequency of T cells to multiple antigenic peptide epitopes. The Assay measures intracellular Ca²⁺ signaling in individual T cells. T cells are labeled with Ca²⁺ sensitive fluorophore and are placed on the glass bottom of a well-covered with antibodies against non-stimulatory T cells' surface receptors. A peptide antigen is injected into the well and the peptide binds to MHC molecules on the T-cell surface. Increase in the intracellular concentration of Ca²⁺ in responding T cells leads to rise in cell fluorescence that is detected by fluorescent microscope; wherein the responding T cells are differentiated into four categories; and the response is calculated for each of the four categories of cells. In certain embodiments, we can identify four categories, but only image and calculate response for three categories (excluding non-responding cells).

A method for detection of the frequency of different responding T cells, each responding to multiple antigenic peptide epitopes comprising: coating glass surface with an agent capable to bind either an antibody or other capturing proteins or capturing agent; covering the surface with an antibody or capturing proteins that binds to a receptor on T-cell surface without interfering with Ca²⁺ flux; adding cloned or polyclonal alpha/beta T cells or gamma/delta T cells labeled with Ca²⁺ sensitive fluorophore to the surface to generate monolayer of the T cells; taking first image of the T-cell monolayer to determine a level of background fluorescence in every individual cell; determining a classification for each responding T cell according to one of four response curves; adding a single or multiple peptide epitopes or live target cells presenting potential peptide epitopes to the T-cell monolayer; measuring the level of fluorescence in every individual T cells on the monolayer by taking multiple images over time image of the T-cell monolayer followed by peptide(s) or live target cells addition bearing peptide epitope; quantifying responses of individual T cells in each of the four classes, by subtracting intracellular fluorescence measured after taking the first image from that images acquired after the first image; and averaging the response in each of the four classes to generate a response rate for each of the four classes.

A method for measuring kinetics of Ca²⁺ flux in differentially responding T cells that form monolayer on the glass surface in response to antigenic peptides or live target cells comprising: immobilizing T cells labeled with Ca²⁺ sensitive fluorophore on the glass bottom of a well, covered with capturing antibody or a capturing protein that bind to non-stimulatory T-cell surface receptor; adding to the well a single or multiple peptide epitopes that binds to the cell surface MHC molecules to be presented for recognition by cognate T cells; the stimulatory signal could also be delivered by live target cells that display peptide epitope(s); wherein the recognition of stimulatory of pMHC by the peptide specific T cells leads to increase of intracellular Ca²⁺ level and fluorescence intensity in the responding T cells, which is then identified after the subtracting fluorescence intensity for every T cell before and after the addition of the peptide antigens; scoring each responding T cell into a category selected from three categories including a rapid and sustained T-cell response, an oscillatory response, or a delayed and oscillatory response, and no response; and measuring changes in number of an individual T cells with increased intracellular fluorescence as function of time provides the kinetic curve of the TCR-mediated Ca²⁺ signaling.

A method for calculating the number of responding T cells, having a particular respond pattern, in a sample comprising: coating glass bottom surface of 96-well plates with an agent capable to bind either an antibody or other capturing proteins; washing said plates free of unbound reagents, wherein the plates were covered with an antibody or other capturing proteins specific for non-stimulatory receptor on the T-cell surface that do not interfere with the induction of T-cell response; blocking the plates with BSA solution; capturing cloned T cell or freshly purified T cells from donor's PBMC labeled with Ca²⁺ sensitive fluorophore; measuring background of intracellular fluorescence for every cell by means of wide field fluorescent microscopy; adding to the wells an antigenic peptide of interest or live target cell presenting potential peptide epitope; measuring fluorescence intensity for every cells in the same fields before and after addition of the stimuli at several time points; stimulating cells with ionomycin and non-stimulatory or “self” peptides serve as positive and negative controls, respectively; calculating the number of cells responding to one of four categories, selected from: a rapid and sustained T-cell response, an oscillatory response, or a delayed and oscillatory response; and comparing intracellular fluorescence in individual cells before and after peptide or live target cells injection using MetaMorph software wherein the number of cells that remain fluorescent in each analyzed field are quantified to calculate the total number of the responding cells per 10⁶ cloned T cells or donor's PBMC.

A method to characterize cell surface markers on T cells with the specificity of interest in order to determine a stage of T-cell differentiation comprising: Immobilizing freshly isolated CD8 T cells labeled with Ca²⁺ sensitive fluorophore and antibodies labeled with non-overlapping fluorophore against cell surface markers of interest on the glass bottom of a well, covered with capturing antibody or a capturing protein that bind to non-stimulatory T-cell surface receptor; measuring background intracellular fluorescence for every cell of the T-cell monolayer and detecting individual T cells that express cell surface markers of interest by means of wide field fluorescent microscopy; calculating total number of cells having an expression pattern according to one of four categories selected from: a rapid and sustained T-cell response, an oscillatory response, or a delayed and oscillatory response, and no responding cells, that express cell surface markers of interest per 10⁶ cloned CD8 T cells or donor's PBMC; adding to the wells an antigenic peptide(s) to be tested; comparing intracellular fluorescence in individual cells before and after peptide injection using MetaMorph software wherein the number of cells that remain fluorescent in each analyzed field are quantified to calculate the total number of the responding CD8 T cells per 10⁶ cells; and calculating the fractions of responding cells in each of four categories that do or do not express surface markers of interest.

In a further embodiment, disclosed is a method for measuring the frequency of responding T cells with the specificity of interest using live target cells presenting peptide(s) of interest or nanoparticles carrying soluble peptide-MHC ligands or any other peptide-MHC oligomers to stimulate T cells recognizing these ligands comprising: Immobilizing T cells labeled with Ca²⁺ sensitive fluorophore on the glass bottom of a well, covered with capturing antibody or a capturing protein that bind to non-stimulatory T-cell surface receptor; measuring background of intracellular fluorescence for every cell of the T-cell monolayer by means of wide field fluorescent microscopy; adding to the wells live target cell presenting peptide(s) of interest or nanoparticles bearing various peptide-MHC or any other peptide-MHC oligomers that ought to be tested; measuring fluorescence intensity for every cells in the same fields after the exposure of T cells in the T-cell monolayer to the above stimuli; determining a category for response for each of the responding cells, according to one of four categories selected from: including a rapid and sustained T-cell response, an oscillatory response, or a delayed and oscillatory response, and non responding; comparing intracellular fluorescence in individual cells before and after the stimulation using MetaMorph software wherein the number of cells that remain fluorescent in each analyzed field are determined as responding cells; and calculating the total number of each type of the responding cells per 10⁶ cloned T cells or donor's PBMC.

A further embodiment is directed to a method to determine the frequency and functional activity of each of four types of antigen-specific CD8 T cells from human PBMC through an assay based on measurement of T-cell intracellular Ca²⁺ signaling induced in response to antigen recognition by T-cell receptor comprising: immobilizing freshly isolated CD8 T cells from human PBMC either intact or labeled with Ca²⁺ sensitive fluorophore on the glass bottom of a well, covered with capturing antibody or a capturing protein that bind to non-stimulatory T-cell surface receptor; adding to the wells unlabeled or fluorescently labeled peptide-MHC proteins assembled on nanoparticles or any other peptide-MHC oligomers to detect antigen-specific T cells and/or to induce Ca²⁺ signaling in the responding T cells; wherein the recognition of unlabeled stimulatory pMHC by the specific T cells labeled with Calcium fluorophore leads to increase of intracellular Ca²⁺ level and fluorescence intensity in the responding T cells, determine which of four classes the responding cells belong to: a rapid and sustained T-cell response, an oscillatory response, or a delayed and oscillatory response, and subtracting fluorescence intensity for every T cell measured before the addition of the stimulatory peptide-MHC oligomers. The binding of fluorescently labeled cognate pMHC to unlabeled T cells will identify both responding and non-responding T cells specific for the same peptide-MHC ligands; and calculating a fraction of responding T cells with the specificity of interest.

In a further embodiment, we compared 3D plot images illustrating Ca²⁺ responses of the T cells to a strong agonist and non-stimulatory peptide to reiterate the ability of the assay to detect responding T cells. In addition, we compared 3D plot images illustrating Ca²⁺ responses of the T cells at high (10⁻ M) and suboptimal (10⁻⁸ M) peptide concentrations. The comparison showed that the decrease of the response magnitude at lower peptide concentration was due to lower amplitude of the responses of individual cells, but not due to the changes in the number of the responding cells.

A further embodiment comprises an express method allowing measuring kinetics of Ca²⁺ flux in responding to antigenic peptides on T cells that form monolayer on the glass surface. Freshly isolated T cells labeled with Ca²⁺ sensitive fluorophore are immobilized on the glass bottom of a well-covered with non-stimulatory antibody specific for a cell surface receptor. Peptides of interest that are added to the T cell monolayer bound to the MHC molecules presented for recognition by cognate T cells. The recognition of stimulatory of pMHC by the peptide specific T cells leads increase of Ca²⁺ and fluorescence intensity in the responding T cells, which could then be identified after the subtracting fluorescence intensity for every T cell before and after the addition of the peptide antigens. Measurements of the number of responding cells as a function of time characterize the kinetics of the Ca²⁺ flux in responding T cells. However, it is necessary to characterize the responding T-cells according to the Figures herein, so as to determine a class of each cell. Accordingly, a time plot can be utilized to determine the kinetics of the Ca²⁺ response for a particular antigen and to determine certain responding cells having characteristics that are suitable for use as therapeutic stem cells for a patient.

A further embodiment is directed to a method of calculating the response rate of a T cells comprising: coating a glass bottom plates with Poly-L-Lysine and, after washing free of unbound reagents, the plates were covered with antibody specific for non-stimulatory receptor on the surface T cells that do not interfere with T cell responses. The plates were blocked with BSA solution prior to addition of T cells. We utilize cloned T cell or freshly purified T cells from donor's PBMC labeled with Ca²⁺ sensitive fluorophore. The capturing of the T cells by the immobilized antibody was facilitated by brief centrifugation at 200 g and unbound cells were removed by gentle washing. We analyze the quality of the T-cell monolayer, which formed on the glass surface, and measured background intracellular fluorescence for every cell by means of wide field fluorescent microscopy. We then add to the wells an antigenic peptide of interest to be tested and measure fluorescence intensity for every cells in the same fields as before at several time points. Stimulating cells with ionomycin and non-stimulatory or “self” peptides serve as positive and negative controls, respectively. Determine the class of cell response according to FIG. 1. We then compare cellular fluorescence in individual cells before and after peptide injection using MetaMorph software. This allow us to quantify the number of cells that remain fluorescent in each analyzed field and to calculate the total number of the responding cells per 10⁶ cloned T cells or donor's PBMC.

A method for detection of the frequency of T cells to multiple antigenic peptide epitopes comprising: Coating a well with poly-L-Lysine or, in further embodiments, optically clear plastic surface can be used that is modified with other chemical agents capable to bind antibodies or other capturing proteins; capturing TS2/4 antibody with said poly-L-Lysine, or in further embodiments, streptavidin can be utilized to capture biotinylated antibody. Furthermore, any other capturing molecules specific to T cell's surface that do not interfere with Ca2+ flux can be utilized; thereafter, adding cloned CD8 T cells with known specificity (OR polyclonal CD8 T cells) and labeling each with Ca²⁺ fluorophore Fluo-4 and adding the T cells to the wells, or, in further embodiments, other cells, including CD4 T cells or gamma/delta T cells can be added to the wells, or, in further embodiments, the cells can be labeled with any Ca2+ sensitive fluorophore and wherein changes in bioelectric properties of T cells can be measured. Finally, you measure the fluorescence and determine classes of response based on the rate and pattern of response of the responding cells. This information can then be utilized to determine whether a particular cell line has the predetermined proper response rate for a particular treatment.

A method for quantification of T cells specific to multiple antigenic peptide epitopes or other ligand recognizable by T cells. In certain embodiments, quantification can be live target cells loaded with peptides, or any antigen presenting cells that display naturally processed peptides such as tumor associated antigen. Furthermore, it could be unknown antigens presented at the cell surface.

A method for determining functional T cell quality by analyzing Ca²⁺ response over time, based on the parameters of any one of the methods described above. In certain embodiments, the analysis may be of T cell surface markers or cytokine (intracellular or released) or analysis of TCR sequences of the responding cells.

These methods are particularly suited for analyzing frequency of T cells recognizing tumor associated antigens within tumor infiltrating lymphocytes (TIL) using either tumor-associated peptide epitopes or antigen-presenting cells sensitized with tumor associated peptide epitopes or live tumor cells. In addition, to measuring frequency of CD8 T cells with desired specificity, the frequency of CD4 T cells recognizing peptide-MHC-II ligands will also be measured.

A method for determining the efficiency of pathogen-specific T cells comprising: preparing a continuous monolayers of freshly isolated T cells labeled with Ca2+ sensitive fluorophore; adding a suspension of tumor cells could be used to detect and isolate tumor specific T cells within the monolayer; measuring Ca2+ responding T cells in the monolayer and to measure the kinetics of Ca2+ flux; and determining the frequency and efficiency of pathogen-specific or tumor-specific T cells within the monolayers.

In each of the embodiments described above, wherein the frequency and efficiency of pathogen-specific or tumor-specific T cells are compared to a control. In certain embodiments, the control is derived from a sample of T cells from a health patient. In certain other embodiments, the control is a predetermined number derived from a plurality of samples from healthy patients.

A method for predicting efficacy of a treatment and a clinical outcome comprising: analyzing the frequency and the efficiency of the responding T cells; wherein said frequency and efficiency will provide an essential information regarding status of the immune response against pathogens or cancer in order to predict the outcome of the infection or cancer spread as well as to choose appropriate treatment for tested individuals; wherein the latter will have significant impact on the cost of treatment and will increase survival rate of the patients. In particular embodiments of the method, the frequency and efficiency of the responding T cells are compared to a control; wherein an efficiency within one standard deviation of the control indicates a functioning immune system; and wherein an efficiency is reduced by more than one standard deviation of the control indicates a compromised immune system. In certain embodiments, indication of a compromised immune systems requires administering to a patient with said comprised immune system a composition suitable for treating a suspected virulent such as CMV.

In preferred embodiments, it is advantageous to utilize a software program that allows for repeated exposures and for automatic calculations of cells based on a set of criteria. To perform the method, a criteria would be set to identify and characterize each individual cell into one of four categories. After characterization, each image is then individually calculated and the cells for each group are split/organized into their various groupings. The software can then mark or identify each cell and fluorescence intensity can be captured without having to resort to manual counting of each cell.

Accordingly, a method comprises immobilizing cells, as provided herein, capturing a background image of all cells; providing an agonist to the cells; capturing a plurality of images of the cells over a time period of X, assigning a value to each of four categories of response and identifying each individual cell within the image as corresponding to one of the four categories; in each image over time period X, averaging response and timing for each of the four categories to determine a response of the cells to the particular agonist.

### Materials and Methods

The materials and methods for generating the underlying layers of T cells are similar to those as described in PCT/US2016/039313, which is incorporated herein by reference in its entirety.

### T Cell Clones and Their Maintenance

HIV- and Flu-specific human CD8+ T cell clones, termed 68A62 and CER43, were kindly provide by Bruce Walker and Antonio Lanzavecchia, correspondingly. These T cells recognize ILKEPVHGV (IV9) and GILGFVFTL (GL9) peptides, respectively, both presented by HLA-A2 MHC class I. 115iX is a CD8+ T cell line developed from CTL D3 as a result of spontaneous mutation in its TCR β chain resulting in loss of specificity for its natural ligand (Anikeeva and Sykulev, unpublished). These were used as T cells with irrelevant specificity. After stimulation with a mixture of allogeneic PBMC and IL2 the cells are typically used in resting stage, 12-17 days after the stimulation as previously described.

Labeling of T Cells with Calcium Indicator

10⁶ cells in 1 ml of PBS were loaded with Fluo-4 (Life Technologies) at 2-4 μM for 30 min at 37° C. in the presence of 0.02% pluoronic acid F-127 and 4 mM Probenecid. The cells were washed free of unreacted reagents and incubated at 37° C. for additional 30 min. The cells were then re-suspended in the assay buffer (Dulbecco's PBS containing 1 mM CaCl2, 2 mM MgCl2, 5 mM glucose, and 0.025% BSA) and used for Ca2+ flux analysis.

### Magnetic Sorting of CD8+ T Cell

CD8 T cells were purified from frozen PBMC using MACS Cell Separation Technology according to manufacturer instruction (Miltenyi Biotec).

### Antibodies and Peptides

Hybridoma producing TS2/4 anti-LFA-1 antibodies was purchased from ATCC. The antibody was purified from culture supernatant by affinity chromatography on protein A Sepharose as described elsewhere. GL9 peptide from the influenza matrix protein was synthesized by Research Genetics, Inc. and IV9 peptide from HIV reverse transcriptase Tsomides, 1991 #167 was a gift from Herman Eisen (MIT).

### Preparation of T-Cell Monolayers

Glass bottom of 96 well MatTec plates was covered with poly-L-Lysine (Sigma, mol wt>300,000) at 0.1 mg/ml for 1 hr at room temperature. After washing with DPBS and add water, TS2/4 non-blocking mAb specific for LFA-1 were added to the plate at concentration 10 μg/ml overnight & 4° C. The wells were washed with DPBS, and 3×10⁵ Fluo-4-labeled T cells in 100 μl of the assay buffer were added to each well. The plates were centrifuged for 200 g for one minute and were incubated for 30 min at room temperature prior to the imaging. Suspended cells were removed by gentle washing with assay buffer. The quality of T cell monolayer was assessed using bright field microscopy.

### Induction and Measurements of Ca²⁺ Flux in T Cells

To identify responding T cells we imaged T-cell monolayers before (background measurement) and after (response) addition of the stimulatory signal such as agonist peptide. The images of T-cell monolayers were taken at various exposure times using 10× or 20× objectives. Instead of averaging the entire set of stimulatory signal against an agonist peptide, we identified sets of cells, having different response rates and then averaged these, now separated sets of cells. This is in direct contrast to the general averaging strategy provided in PCT/US2016/039313. In some experiments Ca2+ response was also initiated by ionomycin at 10 μg/ml to optimize exposure time (data not shown). The average intensity of images prior and after the T-cell stimulation at various time points and the numbers of individual responding cells per imaging field were determined by MetaMorph software.

### Cytolytic Assay

Lymphoblastoid target cells JY (5×10³) were washed, ⁵¹Cr-labeled and then sensitized for one hour with various amounts of a peptide of interest in 150 μl R10 (RPMI-1640 containing 10% FCS). 68A62 CTL in 50 μl in of R10 were then added with a final assay volume of 200 μl. The assay was performed in 96-well round-bottomed plates at an effector-to-target ratio of 5:1. The plates were incubated for four hours in a CO₂ incubator at 37° C. and ⁵¹Cr release was measured in 100 μl of supernatant from each well. Percent specific lysis was determined as previously described in Sykulev, 1996 #209, Anikeeva, 2006 #1422; Beal, 2008 #2027.

### T-Cell Monolayer

Glass bottom of 96-well plates was covered with poly-L-Lysine to capture TS2/4 antibody recognizing LFA-1 adhesion receptor without blocking LFA-1 functional activity. Cloned CD8 T cells with known specificity or polyclonal CD8 T cells were labeled with Ca2+ fluorophore Fluor-4 and added to the wells. The quality of the T-cell monolayers was evident from analysis of bright field images of the immobilized T cells on the glass bottom of the plate. This method allows T cells to form a continuous monolayer allowing the T cells to contact each other, which is necessary for presentation and recognition of pMHC on one T cell by another T cell.

The immobilized cells, after they are provided with stimulation of a peptide, can be imaged. Accordingly, we can take a first image, to identify the background image of each individual cell, before and after the stimulation. Accordingly, we can look at the calcium concentration of each individual cell across time. By looking at each cell, we can then determine and add them to one of three boxes for responding cells, and then a fourth type of cell that is either non-responsive or there is calcium flux that happens with or without stimulation-this typically results in a sharp peak of intensity that quickly diminishes.

### Induction and Analysis of Responding T Cells

To optimize the conditions of the assay, we utilized human CTL clones CER43 and 68A62 recognizing nucleoprotein-derived peptide GL9 from Influenza virus and HIV RT-derived peptide IV9, correspondingly (refs). Both peptides presented to these CTLs are restricted by HLA-A2 protein.

Accordingly, these methods provide for the use of an assay in detecting response of responding T cells to an agonist, visualizing said response, and characterizing the response of said cells to evaluate the agonist, T cells, or both.

What is claimed is:

1. An assay for detecting and quantification of the frequency of T cells to multiple antigenic peptide epitopes wherein the Assay measures intracellular Ca²⁺ signaling in individual T cells; the T cells are labeled with Ca²⁺ sensitive fluorophore and are placed on the glass bottom of a well-covered with antibodies against non-stimulatory T cells' surface receptors; A peptide antigen is injected into the well and the peptide binds to MHC molecules on the T-cell surface;
an Increase in the intracellular concentration of Ca²⁺ in responding T cells leads to rise in cell fluorescence that is detected by fluorescent microscope; wherein the responding T cells are differentiated into at least three categories; and the response is calculated for each of the three categories of cells.

2. The method of claim 1, wherein the at least three categories including a rapid and sustained T-cell response, an oscillatory response, or a delayed and oscillatory response.

3. The method of claim 2, including a fourth category which is a non-responsive T cell.

4. A method for detection of the frequency of different responding T cells, each responding to multiple antigenic peptide epitopes comprising: coating glass surface with an agent capable to bind either an antibody or other capturing proteins; covering the surface with an antibody or capturing proteins that binds to a receptor on T-cell surface without interfering with Ca²⁺ flux; adding cloned or polyclonal T cells or alfa/beta T cells labeled with Ca²⁺ sensitive fluorophore to the surface to generate monolayer of the T cells; taking first image of the T-cell monolayer to determine a level of background fluorescence in every individual cell; determining a classification for each responding T cell according to one of four response curves; adding a single or multiple peptide epitopes or live target cells presenting potential peptide epitopes to the T-cell monolayer; measuring the level of fluorescence in every individual T cells on the monolayer by taking second image of the T-cell monolayer followed by peptide(s) or live target cells addition bearing peptide epitope; quantifying responses of individual T cells in each of the four classes, by subtracting intracellular fluorescence measured after taking the first image from that acquired after the second image; and averaging the response in each of the four classes to generate a response rate for each of the four classes.

5. A method for measuring kinetics of Ca²⁺ flux in differentially responding T cells that form monolayer on the glass surface in response to antigenic peptides or live target cells comprising: immobilizing T cells labeled with Ca²⁺ sensitive fluorophore on the glass bottom of a well, covered with capturing antibody or a capturing protein that bind to non-stimulatory T-cell surface receptor; adding to the well a single or multiple peptide epitopes that binds to the cell surface MHC molecules to be presented for recognition by cognate T cells; the stimulatory signal could also be delivered by live target cells that display peptide epitope(s); wherein the recognition of stimulatory of pMHC by the peptide specific T cells leads to increase of intracellular Ca²⁺ level and fluorescence intensity in the responding T cells, which is then identified after the subtracting fluorescence intensity for every T cell before and after the addition of the peptide antigens; scoring each responding T cell into a category according to three categories including: a rapid and sustained T-cell response, an oscillatory response, or a delayed and oscillatory response; and measuring changes in number of an individual T cells with increased intracellular fluorescence as function of time provides the kinetic curve of the TCR-mediated Ca²⁺ signaling.

6. A method for calculating the number of responding T cells, having a particular respond pattern, in a sample comprising: coating glass bottom surface of 96-well plates with an agent capable to bind either an antibody or other capturing proteins; washing said plates free of unbound reagents, wherein the plates were covered with an antibody or other capturing proteins specific for non-stimulatory receptor on the T-cell surface that do not interfere with the induction of T-cell response; blocking the plates with BSA solution; capturing cloned T cell or freshly purified T cells from donor's PBMC labeled with Ca²⁺ sensitive fluorophore; measuring background of intracellular fluorescence for every cell by means of wide field fluorescent microscopy; adding to the wells an antigenic peptide of interest or live target cell presenting potential peptide epitope; measuring fluorescence intensity for every cells in the same fields before and after addition of the stimuli at several time points; stimulating cells with ionomycin and non-stimulatory or “self” peptides serve as positive and negative controls, respectively; calculating the number of cells responding to one of four categories: a rapid and sustained T-cell response, an oscillatory response, a delayed and oscillatory response, or a non-responding cell; and comparing intracellular fluorescence in individual cells before and after peptide or live target cells injection using MetaMorph software wherein the number of cells that remain fluorescent in each analyzed field are quantified to calculate the total number of the responding cells per 10⁶ cloned T cells or donor's PBMC.

7. A method to characterize cell surface markers on T cells with the specificity of interest in order to determine a stage of T-cell differentiation comprising: Immobilizing freshly isolated CD8 T cells labeled with Ca²⁺ sensitive fluorophore and antibodies labeled with non-overlapping fluorophore against cell surface markers of interest on the glass bottom of a well, covered with capturing antibody or a capturing protein that bind to non-stimulatory T-cell surface receptor;
measuring background intracellular fluorescence for every cell of the T-cell monolayer and detecting individual T cells that express cell surface markers of interest by means of wide field fluorescent microscopy; calculating total number of cells having an expression pattern according to one of three categories including rapid and sustained T-cell response, an oscillatory response, or a delayed and oscillatory response, that express cell surface markers of interest per 10⁶ CD8 T cells; adding to the wells an antigenic peptide(s) to be tested; comparing intracellular fluorescence in individual cells before and after peptide injection using MetaMorph software wherein the number of cells that remain fluorescent in each analyzed field are quantified to calculate the total number of the responding CD8 T cells per 10⁶ cells; and calculating the fractions of responding cells in each of the three categories that do or do not express surface markers of interest.

8. A method for measuring the frequency of responding T cells with the specificity of interest using live target cells presenting peptide(s) of interest or nanoparticles carrying soluble peptide-MHC ligands or any other peptide-MHC oligomers to stimulate T cells recognizing these ligands comprising: Immobilizing T cells labeled with Ca²⁺ sensitive fluorophore on the glass bottom of a well, covered with capturing antibody or a capturing protein that bind to non-stimulatory T-cell surface receptor; measuring background of intracellular fluorescence for every cell of the T-cell monolayer by means of wide field fluorescent microscopy; adding to the wells live target cell presenting peptide(s) of interest or nanoparticles bearing various peptide-MHC or any other peptide-MHC oligomers that ought to be tested; measuring fluorescence intensity for every cells in the same fields after the exposure of T cells in the T-cell monolayer to the above stimuli; determining a category for response for each of the responding cells, according to one of three categories, including rapid and sustained T-cell response, an oscillatory response, or a delayed and oscillatory response; comparing intracellular fluorescence in individual cells before and after the stimulation using MetaMorph software wherein the number of cells that remain fluorescent in each analyzed field are determined as responding cells; and calculating the total number of each type of the responding cells per 10⁶ cloned T cells or donor's PBMC.

9. A method to determine the frequency and functional activity of each of four types of antigen-specific CD8 T cells from human PBMC through an assay based on measurement of T-cell intracellular Ca²⁺ signaling induced in response to antigen recognition by T-cell receptor comprising: immobilizing freshly isolated CD8 T cells from human PBMC either intact or labeled with Ca²⁺ sensitive fluorophore on the glass bottom of a well, covered with capturing antibody or a capturing protein that bind to non-stimulatory T-cell surface receptor; adding to the wells unlabeled or fluorescently labeled peptide-MHC proteins assembled on nanoparticles or any other peptide-MHC oligomers to detect antigen-specific T cells and/or to induce Ca²⁺ signaling in the responding T cells; wherein the recognition of unlabeled stimulatory pMHC by the specific T cells labeled with Calcium fluorophore leads to increase of intracellular Ca²⁺ level and fluorescence intensity in the responding T cells, determine which of four classes the responding cells belong to: a rapid and sustained T-cell response, an oscillatory response, a delayed and oscillatory response, or a non-responding cell, and subtracting fluorescence intensity for every T cell measured before the addition of the stimulatory peptide-MHC oligomers. The binding of fluorescently labeled cognate pMHC to unlabeled T cells will identify both responding and non-responding T cells specific for the same peptide-MHC ligands; and calculating a fraction of responding T cells with the specificity of interest.

10. A method allowing measurement of kinetics of Ca²⁺ flux in responding to antigenic peptides on T cells that form monolayer on the glass surface of a variety of differentially responding T cells; comprising: fixing freshly isolated T cells labeled with Ca²⁺ sensitive fluorophore are immobilized on the glass bottom of a well-covered with non-stimulatory antibody specific for a cell surface receptor.; adding Peptides of interest that are added to the T cell monolayer bound to the MHC molecules presented for recognition by cognate T cells; increasing the recognition of stimulatory of pMHC by the peptide specific T cells leads increase of Ca²⁺ and fluorescence intensity in the responding T cells, which could then be identified after the subtracting fluorescence intensity for every T cell before and after the addition of the peptide antigens; measuring the number of responding cells as a function of time characterize the kinetics of the Ca²⁺ flux in responding T cells; characterizing the responding T-cells according to one of four classes of response, so as to determine a class of each cells; plotting a time plot to determine the kinetics of the Ca²⁺ response for a particular antigen and to determine certain responding cells having characteristics that are suitable for use as therapeutic stem cells for a patient.

11. A method of calculating the response rate of a T cells comprising: coating a glass bottom plates with Poly-L-Lysine and, after washing free of unbound reagents, the plates were covered with antibody specific for non-stimulatory receptor on the surface T cells that do not interfere with T cell responses; blocking the plates with BSA solution prior to addition of T cells. We utilize cloned T cell or freshly purified T cells from donor's PBMC labeled with Ca²⁺ sensitive fluorophore; capturing of the T cells by the immobilized antibody was facilitated by brief centrifugation at 200 g and unbound cells were removed by gentle washing; analyzing the quality of the T-cell monolayer, which formed on the glass surface, and measured background intracellular fluorescence for every cell by means of wide field fluorescent microscopy; adding to the wells an antigenic peptide of interest to be tested and measure fluorescence intensity for every cells in the same fields as before at several time points; stimulating cells with ionomycin and non-stimulatory or “self” peptides serve as positive and negative controls, respectively;
determining the class of cell response: including a rapid and sustained T-cell response, an oscillatory response, or a delayed and oscillatory response; comparing cellular fluorescence in individual cells before and after peptide injection using MetaMorph software; quantifying the number of cells that remain fluorescent in each analyzed field and to calculate the total number of the responding cells per 10⁶ cloned T cells or donor's PBMC.

12. A method for detection of the frequency of T cells to multiple antigenic peptide epitopes comprising: Coating a well with poly-L-Lysine or, in further embodiments, optically clear plastic surface can be used that is modified with other chemical agents capable to bind antibodies or other capturing proteins; capturing TS2/4 antibody with said poly-L-Lysine, or in further embodiments, streptavidin can be utilized to capture biotinylated antibody. Furthermore, any other capturing molecules specific to T cell's surface that do not interfere with Ca2+ flux can be utilized; thereafter, adding cloned CD8 T cells with known specificity (OR polyclonal CD8 T cells) and labeling each with Ca²⁺ fluorophore Fluo-4 and adding the T cells to the wells, or, in further embodiments, other cells, including CD4 T cells or gamma/delta T cells can be added to the wells, or, in further embodiments, the cells can be labeled with any Ca2+ sensitive fluorophore and wherein changes in bioelectric properties of T cells can be measured. Finally, you measure the fluorescence and determine classes of response based on the rate and pattern of response of the responding cells. This information can then be utilized to determine whether a particular cell line has the predetermined proper response rate for a particular treatment.

13. A method for determining the efficiency of pathogen-specific T cells comprising: preparing a continuous monolayers of freshly isolated T cells labeled with Ca2+ sensitive fluorophore; adding a suspension of tumor cells could be used to detect tumor specific T cells within the monolayer; measuring Ca2+ responding T cells in the monolayer and to measure the kinetics of Ca2+ flux; and determining the frequency and efficiency of pathogen-specific or tumor-specific T cells within the monolayers.

14. A method for predicting efficacy of a treatment and a clinical outcome comprising: analyzing the frequency and the efficiency of the responding T cells; wherein said frequency and efficiency will provide an essential information regarding status of the immune response against pathogens or cancer in order to predict the outcome of the infection or cancer spread as well as to choose appropriate treatment for tested individuals; wherein the latter will have significant impact on the cost of treatment and will increase survival rate of the patients; charactering the response rate of each responding cell into one of at least four groups; averaging the response of the cells in each group as compared to the control; plotting the response of each group over time; wherein an efficiency within one standard deviation of the control indicates a functioning immune system; and wherein an efficiency is reduced by more than one standard deviation of the control indicates a compromised immune system.
